(19) United States
(12) Patent Application Publication
(10) Pub. No.: US 2026/0151314 A1
(43) Pub. Date: Jun. 4, 2026

(54) PERSONAL CARE COMPOSITION

(71) Applicant: THE PROCTER & GAMBLE COMPANY, Cincinnati, OH (US)

(72) Inventors: Jessa Leigh MEYERS, Cincinnati, OH (US); Toni-Josephine Yamzon ESCUADRO, Cincinnati, OH (US); Sumanth Narahari JAMADAGNI, West Chester, OH (US); Debora W. CHANG, Mason, OH (US)

(21) Appl. No.: 19/407,173

(22) Filed: Dec. 3, 2025

Related U.S. Application Data
(60) Provisional application No. 63/727,284, filed on Dec. 3, 2024.

(51) Int. Cl. A61K 8/44; A61K 8/19; A61K 8/365; A61K 8/46; A61Q 5/02; A61Q 5/12; A61Q 19/00; A61Q 19/10

(52) U.S. Cl. CPC A61K 8/442; A61K 8/19; A61K 8/365; A61K 8/466; A61Q 5/02; A61Q 5/12; A61Q 19/007; A61Q 19/10; A61K 2800/596

(57) ABSTRACT

An aqueous personal care composition that includes an anionic surfactant and an amphoteric or zwitterionic surfactant. The amphoteric or zwitterionic surfactant typically includes a betaine. The composition has a pH of about 3.0 to about 6.5 such that the amphoteric or zwitterionic surfactant is protonated, which helps provide a consumer-preferred viscosity of 2,500 mPa-s to 25,000 mPa-s, along with a variety of other benefits.

### CROSS REFERENCE TO RELATED APPLICATIONS

This application claims the benefit, under 35 U.S.C. § 119(e), to U.S. Provisional Application No. 63/727,284, filed Dec. 3, 2024, the entire disclosure of which is fully incorporated by reference herein.

### FIELD OF THE INVENTION

The present disclosure generally relates to a personal care composition that includes an anionic surfactant and an amphoteric or zwitterionic surfactant and has a pH of less than 7. More specifically, the present disclosure relates to a personal care composition that includes a sulfate-free anionic surfactant, a betaine co-surfactant, and has a pH of less than 7 and a desirable viscosity.

### BACKGROUND

Rheology is an important factor in personal care formulations because it affects consumer acceptance as well as product performance. The selection, total amount, and ratio of surfactants contributes to the rheology of a personal cleansing composition. Sulfated surfactants like sodium lauryl sulfate (SLS) and sodium laureth sulfate (SLES) are commonly used in personal cleansing composition due to their good cleaning and lather performance. Inorganic salt such as sodium chloride (NaCl) is commonly used to adjust the viscosity of personal care compositions containing SLS and SLES. However, when sulfate surfactants are eliminated, building the desired rheological properties can be challenging.

Typical levers used in sulfate-free surfactant compositions include, for example, increasing total surfactant amount, adding polymeric thickeners, and adding simple salts. But these approaches have drawbacks. For example, increasing total surfactant amount can be costly and form higher ordered phases that “trap” surfactant, impairing foamability. Adding polymeric thickeners can have an unwanted non-Newtonian gelation effect that does not dilute and spread easily. And inorganic salt can introduce instability in the composition. In previous studies, it has been found that Alpha Olefin Sulfonate (AOS) and cocamidopropyl betaine (CAPB) can afford viscosity builds with salt addition.

Crystallization of surfactants in cold temperature is due to the low solubility of the surfactants, which is exacerbated by excess salt due to the “common-ion” effect. High levels of added salt to thicken by elongating micelles can have the undesirable consequence of salting out lightly charged cationic polymers and/or leading to segregative coacervation. Biodegradable, ethoxylate-free, low molecular weight thickeners are not available, and phase separation has been a challenge with many biodegradable high molecular weight thickeners.

A need, therefore, exists to provide a personal care composition having the desired rheology without such drawbacks.

### SUMMARY

Disclosed herein is a personal care composition, comprising an anionic surfactant comprising an acyl taurate surfactant, an N-alkyl acyl taurate surfactant, and an alpha olefin sulfonate surfactant, or salts thereof; an amphoteric or zwitterionic surfactant; an aqueous carrier; and about 0.3% to about 3% of an inorganic salt thickener. The composition has a pH of about 3.0 to about 6.5 and a viscosity of about 2,500 mPa-s to 25,000 mPa-s.

### BRIEF DESCRIPTION OF THE DRAWINGS

FIG. 1 is a chart summarizing the ln(viscosity) to pH of Inventive Example 2A and Comparative Examples A1 and A2.

FIG. 2 is a chart summarizing the ln(viscosity) to pH of Inventive Example 2B and Comparative Example B.

FIG. 3 is a chart summarizing the ln(viscosity) to pH of Inventive Example 2C and Comparative Examples C1 and C2.

FIG. 4 is a chart summarizing the ln(viscosity) to pH of Inventive Example 2D and Comparative Example D.

FIG. 5 is a chart summarizing the ln(viscosity) to pH of Inventive Examples 2E to 2I.

FIG. 6 is a chart summarizing the ln(viscosity) to pH of Inventive Examples 2J to 2L.

### DETAILED DESCRIPTION

Recent trends indicate a desire by consumers to replace sulfated cleansing compositions with milder, sulfate-free versions. However, when sulfate surfactants are eliminated from a personal care composition like a shampoo or body wash, building the desired rheological properties can be challenging. Surprisingly, it has now been discovered that pH can be used to control the rheology of certain products that include an anionic detersive surfactant and an amphoteric or zwitterionic co-surfactant.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the personal care composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Substantially free of” means a composition or ingredient includes less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

“Titratable,” refers to a species or compound whose protonation state changes with pH.

### Personal Care Composition

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, conditioning benefit to hair in combination with a detersive taurate surfactant.

Liquid personal care compositions herein, such as shampoos, conditioners, and body washes may have a viscosity of 2,500 mPa-s to 25,000 mPa-s (e.g., 2,500-15,000 mPa-s, 3,000-10,000 mPa-s or 3,500-9,000 mPa-s) according to the Rheology method described in more detail below. It is believed that viscosities in this range are generally preferred by users of liquid personal care compositions.

The details of the various components will be discussed in detail hereinafter. Generally, however, the personal care compositions may include a detersive surfactant for cleaning a target bodily surface such as hair and skin. The detersive surfactant includes an anionic surfactant such as, for example, a taurate, an alpha olefin sulfonate, an amino acid-based surfactant, salts of these and combinations thereof. In some aspects, the anionic surfactant may may be selected from an acyl taurate, an N-alkyl acyl taurate (e.g., N-methyl, N-ethyl, N-propyl or N-butyl acyl taurate surfactant), a C14-C16 alpha olefin sulfonate, a salts of these, and combinations thereof. In some embodiments, the anionic detersive surfactant may be titratable. Some non-limiting examples of titratable anionic surfactants include glutamates and alaninates.

The personal care composition may also include one or more co-surfactants, for example, to help solubilize the detersive surfactant or another ingredient in the composition or help improve the foaming characteristics of the composition. The co-surfactants may include an amphoteric surfactant comprising a betaine and/or a zwitterionic surfactant comprising a betaine.

A betaine surfactant has both a positively charged functional group and a negatively charged functional group. The positively charged functional group may be deprotonated, meaning it bears no hydrogen atom. The positively charged functional group may be, for example, a quaternary ammonium or phosphonium cation. The negatively charged functional group may be, for example, a carboxylate group. The negatively charged functional group, such as a carboxylic group, may have a pKa that is much lower than a typical carboxylic group due to the presence of the positively charged functional group, such as an adjacent quaternary nitrogen, that stabilizes the deprotonated state. Non-limiting examples of co-surfactants that may be suitable for use herein include cocamidopropyl betaine (CAPB) and lauramidopropyl betaine (LAPB). The carboxylic group in CAPB/LAPB has a pKa of approximately 2.0, which is lower than a typical carboxylic group (pKa ˜4.5) due to the presence of an adjacent quaternary nitrogen that stabilizes the deprotonated state. It has been discovered that in the presence of an anionic surfactant, the pKa of the carboxylic group increases due to unfavorable interactions between the deprotonated (anionic) carboxylic group and anionic headgroups of the anionic surfactant.

The total surfactant amount in the personal care composition can range from 10% to 30% (e.g., 11% to 24%, 12% to 23%, 13% to 22%, 14% to 21%, 15% to 20%, 16% to 19%, 17% to 18%, 13% to 18%) by weight, based on the weight of the personal care composition. In some aspects of the disclosure, the weight ratio of detersive surfactant to co-surfactant may be 1:1 to 2:1 (e.g., 1.9:1, 1.8:1, 1.7:1, 1.6:1, 1.5:1, 1.4:1, 1.3:1, 1.2:1, or even 1.1:1). It is to be appreciated that the surfactants referred to herein also include their corresponding salts (e.g., sodium, potassium, magnesium, ammonium, or triethanolamine).

The personal care composition may further include an aqueous carrier. The personal care composition may include a polymeric thickener, including a low charge density polymer to aid in the appearance and/or feel of hair. The personal care compositions herein may be sulfate-free. When sulfate surfactants are eliminated, building the desired rheological properties can be challenging.

It has been discovered that pH may be used to control the rheology of the personal care composition herein. More specifically, it has been discovered that the carboxylic acid group in a betaine surfactant may be protonated by providing the personal care compositions with a pH in a range of 3.0 to 6.5 (e.g., 4.0 to 6.0 or 4.5 to 5.5). When the carboxylic group is protonated, the zwitterionic betaine headgroup becomes cationic resulting in favorable cationic-anionic interactions between betaine and anionic surfactant headgroups. The favorable ionic interactions yields much better molecular packing of surfactants, a reduction of micelle scission energies, and longer micelles, which provide a higher viscosity.

Personal care compositions with a pH in a range of 3.0 to 6.5 may result in numerous other benefits. Crystallization of surfactants in cold temperature is due to the low solubility of the surfactants, which is exacerbated by excess salt due to the ‘common-ion’ effect. The low pH ranges may also improve cold-temperature stability and may provide some scalp/skin health benefits.

High levels of added salt to thicken by elongating micelles can have the undesirable consequence of salting out lightly charged cationic polymers and/or leading to segregative coacervation. Low pH may reduce or eliminate the amount of inorganic salt thickeners needed to build viscosity, which enables the use of low charge density polymers and improves cold-temperature stability.

Low pH may also bolster the preservative system, enabling weak organic acid preservatives such as sodium benzoate to become more efficacious. Biodegradable, ethoxylate-free, low molecular weight thickeners are not available, and phase separation has been a challenge with many biodegradable high molecular weight thickeners. Low pH enables progress towards cleaner formulas with fewer components.

Various embodiments may avoid utilizing components that hydrolyze at lower pH, such as certain surfactants, like isethionates and sarcosinates. Some surfactants, such as lauroyl taurates and lauroyl methyl taurates have been shown to be robust against hydrolysis at the low pH (3.0 to 6.5) described according to various embodiments. Some perfumery raw materials (PRMs), such as esters, may also be sensitive to hydrolysis at the pH ranges utilized and may need to be modified, substituted, or avoided.

### Detersive Surfactant

The personal care compositions include a detersive surfactant to provide foaming and cleaning properties. Detersive surfactants facilitate cleaning due to their amphiphilic nature, which allows the surfactants to break up, and form micelles around, oil and other contaminants on the hair. The “entrapped” contaminant can then be rinsed off more easily with water. The detersive surfactant may include one or more anionic surfactants or salts thereof. The detersive surfactant may be present in the personal care composition in an amount of 5% to 20% by weight, based on the weight of the personal care composition. In some aspects, the detersive surfactant may be a titratable anionic surfactant. A titratable anionic surfactant is a surfactant whose protonation state changes with pH (e.g., in the range of pH 3-7) due to the presence of a carboxylate group or a phosphate group. The protonation state and pKa of a titratable surfactant can be measured using titration with an acid or base and fitting the data to the Henderson-Hasselbalch equation or via NMR spectroscopy as a function of pH Examples of titratable anionic surfactants include but are not limited to fatty acid- and amino acid-based surfactants and surfactants that include a carboxylate group and a sulfate or sulfonate group, such as sulfosuccinate surfactants. It is to be appreciated that the personal care compositions herein may be free of or substantially free of sulfate surfactants.

The detersive surfactant may include an acyl taurate surfactant. The acyl taurate surfactants herein may be saturated or unsaturated. Acyl taurate surfactants that may be suitable for use herein are generally described by Formula I below.

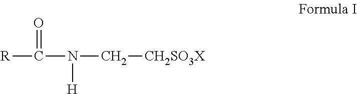

Where: R may be an alkyl group with 5 to 23 carbon atoms (e.g., 7-21, 7-17, 7-15, 7-13, 11-17, 11-15, 11-13 or even 11 carbon atoms) and X may be a suitable counterion (e.g., sodium, potassium, magnesium, ammonium or triethanolamine).

Some nonlimiting examples of acyl taurates are capric ester taurate, cocoyl taurate, lauroyl taurate, myristoyl taurate, caproyl taurate, oleoyl taurate, capryloyl taurate, palmitoyl taurate, stearoyl taurate, linoleoyl taurate, salts of these and combinations thereof. An example of a suitable acyl taurate salt is sodium lauroyl taurate.

The acyl taurate surfactant may be present in the anionic surfactant at an amount of 60% to 90% (e.g., 65% to 85%, 70% to 80%, or even about 75%) by weight, based on the total weight of the anionic surfactant.

The anionic surfactant may include an N-alkyl acyl taurate surfactant. The N-alkyl acyl taurate surfactants herein may be either saturated or unsaturated. The N-alkyl acyl taurate surfactants herein are generally described by Formula II below.

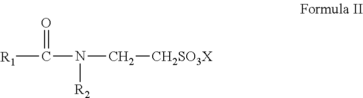

Where: R₁ may be an alkyl group with 5 to 23 carbon atoms (e.g., 7-21, 7-17, 7-15, 7-13, 11-17, 11-15, 11-13 or even 11 carbon atoms) and X may be a suitable counterion (e.g., sodium, potassium, magnesium, ammonium or triethanolamine) and R₂ may be an alkyl group with 1 to 4 carbon atoms. Some nonlimiting examples of N-alkyl acyl taurates that may be suitable for use herein include methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate and combinations thereof. An example of a suitable N-alkyl acyl taurate salt is sodium methyl lauroyl taurate. The N-alkyl acyl taurate surfactant may be present in the at an amount of 1% to 10% (e.g., 2% to 9%, 3% to 8%, 4% to 7% or 5% to 6%) by weight, based on the weight of the anionic surfactant.

Acyl taurate and N-alkyl acyl taurate surfactants used in the personal care compositions herein may be selected from the above examples and combinations thereof.

The anionic surfactant may include an alpha olefin sulfonate surfactant. The alpha olefin sulfonate surfactants herein may be saturated or unsaturated and are generally described by Formula III below.

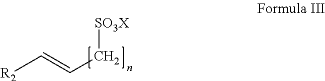

Where: R₃ may be an alkyl group with 5 to 23 carbon atoms (e.g., 7 to 21, 7 to 17, 7 to 15, 7 to 13, 11 to 17, 11 to 15, 11 to 13, 14 to 16, or even 11 carbon atoms) and X may be a suitable counterion (e.g., sodium, potassium, magnesium, ammonium or triethanolamine) and n may be 1 or 2. According to various aspects of the disclosure, the alpha olefin sulfonate surfactant may be a C14 to C16 alpha olefin sulfonate surfactant, in which case R₃ may be comprise from 10 to 13 carbon atoms.

The alpha olefin sulfonate surfactant may be present in the anionic surfactant at an amount of 50% to 75% by weight, based on the weight of the anionic surfactant.

The anionic surfactant may comprise one or more amino acid-based surfactants, such as glycinates, alaninates, and glutamates. Amino-acid based surfactants may offer good foaming properties, contributing to a pleasant cleansing experience, and leave the skin and hair feeling soft and conditioned. The amino acid-based surfactant may be present at an amount of 50% to 75% by weight, based on the weight of the anionic surfactant.

Non-limiting examples of glutamates include lauroyl glutamate, cocoyl glutamate, myristoyl glutamate, palmitoyl glutamate, stearoyl glutamate. Examples of glutamate salts include sodium cocoyl glutamate, disodium cocoyl glutamate, sodium lauroyl glutamate, potassium cocoyl glutamate, triethanolamine cocoyl glutamate, sodium myristoyl glutamate, sodium palmitoyl glutamate, sodium stearoyl glutamate, and combinations thereof.

Non-limiting examples of alaninates include lauroyl alaninate, cocoyl alaninate, myristoyl alaninate, palmitoyl alaninate, stearoyl alaninate, and combinations thereof. Examples of some specific alaninate salts include sodium cocoyl alaninate, sodium lauroyl alaninate, potassium cocoyl alaninate, triethanolamine cocoyl alaninate, sodium myristoyl alaninate, sodium palmitoyl alaninate, sodium stearoyl alaninate.

Non-limiting Examples of glycinates include lauroyl glycinate, cocoyl glycinate, myristoyl glycinate, palmitoyl glycinate, stearoyl glycinate, and combinations thereof. Examples of amino-acid based surfactant salts include sodium cocoyl glycinate, potassium cocoyl glycinate, and sodium lauroyl glycinate.

Amino-acid based surfactants, when included in the personal care composition, may be selected from the above examples or combinations thereof.

The personal care composition may, optionally, include one or more additional anionic surfactants other than those described above. Optional anionic surfactants that may be suitable for use herein include non-taurate, non-sulfate anionic surfactants such as carboxylates, sulfonates (e.g., linear alkylbenzene sulfonates, alkyl glyceryl sulfonates, sodium laurylglucosides hydroxypropylsulfonate), branched alkyl sulfates, sulfosuccinates, sulfoacetates, sulfolaurates, lactate- and lactylate-based surfactants (e.g., sodium lauroyl lactate and sodium lauroyl lactalyte), phosphate ester surfactants, and combinations of these.

### Co-Surfactant

The personal care composition herein may include a co-surfactant in an amount of 1% to 20% (e.g., 2% to 18%, 3% to 15%, 4% to 12% or 5% to 10%) by weight, based on the weight of the personal care composition. The co-surfactants may be selected from amphoteric surfactants, zwitterionic surfactants, non-ionic surfactants and combinations of these. If an amphoteric or zwitterionic surfactant is selected, it may be present at 30% to 50% (e.g., 30% to 45% or 35% to 40%) by weight, based on the weight of the total surfactant in the personal care composition.

The amount of co-surfactant in the composition can be important and should be tailored to balance solubility and/or viscosity building with cleaning and/or conditioning benefit. For example, too much amphoteric co-surfactant can make the surfactant system less salt tolerant and may impede the ability of the surfactant system to form a suitable coacervate upon dilution with water. This can be especially problematic when the composition contains a cationic polymer because the lowered salt tolerance of the surfactant system may cause the cationic polymer to precipitate out. In some embodiments, the composition may include a weight ratio of total detersive surfactant to co-surfactant of 12:1 to 3:10 (6:1 to 3:10, 4:1 to 1:3, or even 2:1 to 1:2).

Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. It is to be appreciated that the terms “zwitterionic” and “amphoteric” may apply to a single compound. For example, the terms both apply to CAPB, because CAPB's amphoteric behavior stems from its zwitterionic structure. The presence of both positive and negative charge centers in CAPB allows it to react as both an acid (by donating a proton in basic conditions) and a base (by accepting a proton in acidic conditions).

In some aspect, the amphoteric and/or zwitterionic surfactant may be a derivative of an aliphatic secondary and tertiary amines in which one of the aliphatic substituents contains from 8 to 18 carbon atoms and one aliphatic substituent contains an anionic group such as a carboxy, sulfonate, phosphate, or phosphonate group. Non-limiting examples of zwitterionic surfactants includes sultaines and betaines. Non-limiting examples of amphoteric surfactants include amphoacetates, amphodiacetates and propionates.

In some aspects, it may be particularly desirable for the co-surfactant to include a betaine.

Some non-limiting examples of betaines that may be suitable include alkyl betaines, alkylamido betaines, amine oxide betaines, sulfobetaines and combinations thereof. Some non-limiting examples of alkyl betaines include cetyl betaine, behenyl betaine, erucyl betaine, dodecyl betaine, tetradecyl betaine, hexadecyl betaine, and octadecyl betaine. Examples of alkylamido betaines include but are not limited to lauramidopropyl betaine (LAPB), myristamidopropyl betaine, palmitamidopropyl betaine, stearamidopropyl betaine, oleamidopropyl betaine, ricinoleamidopropyl betaine, cocoamidopropyl betaine (CAPB), and coco betaine. Examples of amine oxide betaines include but are not limited to lauramine oxide, myristamine oxide, palmitamine oxide, stearamine oxide, and cocoamidoamine oxide. CAPB and LAPB may be particularly suitable co-surfactants for use herein.

In some aspects, the co-surfactant may include a non-ionic surfactant, selected from, for example, glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereof. Some particularly suitable examples of non-ionic surfactants include cocamide, cocamide monoethanolamine (MEA), PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof. Cocamide MEA may be particularly suitable for use as an optional non-ionic surfactant, for example, in an amount of (0.5% to 5% (e.g., 1% to 4%, 1% to 3%, 2 to 3%, less than or equal to 3%, or combinations thereof) by weight, based on the weight of the composition.

Additional examples of optional co-surfactants are disclosed in US 2019/0105246, US 2018/0098923, U.S. Pat. No. 9,271,908, WO 2020/016097, and McCutcheon's Emulsifiers and Detergents, 2019, MC Publishing Co.

Co-surfactant that are suitable for use in the personal care compositions herein may be selected from those exemplified above or combinations thereof.

### Aqueous Carrier

The personal care composition may optionally include 20 to 95% of an aqueous carrier such as water and/or a water miscible solvent. The type and amount of aqueous carrier should be selected to provide the composition with the desired rheological properties. The liquid carrier can be water with, e.g., less than 10%, 7%, 5%, 3%, 1%, 0.5% or even 0% miscible organic solvent.

Some nonlimiting examples of organic solvents include lower alkyl alcohols (e.g., ethanol and isopropanol) and polyhydric alcohols (e.g., propylene glycol, hexylene glycol, glycerin, and propane diol).

### Preservatives

The personal care composition may include a preservative system, In some aspects, the preservative system may include a weak organic acid such as sodium benzoate. The low pH of the personal care composition can bolster the preservative system, enabling a weak organic acids like sodium benzoate to become more efficacious.

### Inorganic Salt Thickener

The personal care composition may be free of or substantially free of an inorganic salt thickener such as sodium chloride, potassium chloride, sodium sulfate, ammonium chloride, sodium bromide, combinations of these and the like. In some sulfate-free compositions, inorganic salt can introduce instability to the composition by aiding in the formation of a coacervate between anionic surfactants and cationic polymers which may be present. The coacervate typically has a gel-like consistency which may appear cloudy or as precipitate layer, and it can impact the rheological and performance properties of the composition as well as the consumer-perceived quality of the product. Thus, it can be important to specifically tailor the amount of inorganic salt in the composition formulation. It can be particularly important to limit the amount of inorganic salt when using cationic polymers that have a molecular weight of 500,000 Da and a charge density of less than 1.0 meq/g, as these polymers tend to be more sensitive to salt than other cationic polymers. Of course, it is to be appreciated that when the cleansing composition is used as intended, it will form a coacervate upon dilution to provide the desired cleaning and/or conditioning benefit.

The low pH of the personal care composition reduces or eliminates the amount of simple salt needed to build viscosity, thereby enabling the use of low charge density polymers and improving cold-temperature stability. Of course, an inorganic salt thickener is not forbidden, and in some aspects, the compositions herein may contain an inorganic salt thickener at a level of0.01% to 5%, (e.g., 0.05% to 1% or 0.1% to 0.5).

### Cationic Polymers

The personal care compositions herein may include 0.05 to 3% of a cationic polymer (e.g., 0.1 to 2%, or even 0.2 to 0.8%) to provide improved appearance, feel or deposition benefits to hair or skin. The cationic polymer can have a weight average molecular weight of 50 kDa to 5 MDa (e.g., 500 kDa to 4 MDa, 1 to 3 MDa, 1.2 to 2 MDa, or even 1.4 to 1.8 MDa) and a charge density of 0.2 meq/g to 12 meq/g (e.g., 0.4 to 10 meq/g, 0.4 to 5 meq/g, 0.4 to 4 meq/g, 0.4 to 3 meq/g, or even 0.4 to 2 meq/g). The charge densities can be measured at the pH of intended use of the personal care composition, which can be pH 3 to pH 9 (e.g., pH 4 to 8 or pH 4.5 to 6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some non-limiting examples of cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some nonlimiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Personal Care Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride (Polyquaternium-76). AM:TRIQUAT may have a charge density of 1.6 meq/g and a molecular weight of 1.1 MDa.

In some aspects, the cationic monomer can be polymethyacrylamidopropyl trimonium chloride, available under the trade name Polycare® 133, from Solvay (Brussels, Belgium).

Copolymers of the cationic monomer may also suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Pat. No. 6,930,078.

In some instances, the cationic polymer may include a synthetic cationic polymer or derivative thereof present at 0.025% to 5%. Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

Cationic polymers suitable for use herein may be selected from those exemplified above or combinations thereof.

### Optional Ingredients

The personal care compositions described herein may include a variety of optional ingredients to tailor the properties and characteristics of the composition, as desired. The optional ingredients may be materials that are commonly included in compositions of the type. The optional ingredients should be physically and chemically compatible with the essential components of the personal care composition and should not otherwise unduly impair the stability, aesthetics, or performance of the composition. Individual concentrations of optional components can generally range from 0.001% to 10%.

Some non-limiting examples of optional ingredients that can be included in the personal care compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Method of Making a Personal Care Composition

The personal care composition described herein can be made using conventional methods for making compositions of the type desired (e.g., shampoo, conditioner or body wash). A particularly suitable method of making the compositions herein is described in Example 1 below. In some aspects, the composition may include a gel network to aid in the conditioning of hair or scalp. U.S. Publication No. 2006/269501 discloses methods of making gel networks that may be suitable for use herein.

### Method of Use

The personal care compositions described herein can be used in a conventional manner for cleansing and conditioning of hair or skin. Effective amounts of the composition for use generally range from 1 g to 50 g (e.g., 1 g to 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid, solid, semi-solid, flake, gel, foam, in a pressurized container with a propellant added, or used in a pump spray form. The viscosity of the product may be selected to accommodate the form desired.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Rheology Method

For viscosity measures above 1,200 cPs, personal care composition viscosities can be measured on a 2.5 mL sample using a cone and plate Brookfield® RS brand rheometer or equivalent with cone C75-1 at 2 s⁻¹, 27° C. at 3 mins. For viscosity measures below 1,200 cPs, personal care composition viscosities can be measures on a ˜15 mL sample (fill sample well so that entire cylinder surface area is exposed to product) using a TA Instruments HR Rheometer or equivalent with concentric cylinder (Couette) geometry at 2 s⁻¹ based on flow sweep at sheer rates between 0.1 s⁻¹ to 100 s⁻¹ at 90 s timesteps, 25° C.

### EXAMPLES

### Example 1: Example Formulations

Table 1 provides examples of inventive personal care composition formulations. The compositions in Table 1 were made by adding DI water to a mixing vessel and then adding each subsequent ingredient while stirring. Surfactants with low water solubility such as cocamide MEA or sodium cocoyl isethionate, if present, require the composition to be heated to 50-75° C. and stirred until fully solubilized (i.e., no visible particles remain and batch is clear). The remaining ingredients, except for cationic polymer or volatile materials, are then added to the mixing vessel and mixed until fully dissolved or solubilized. If heated, the composition is cooled to 35° C. or less before volatile ingredients such as perfume are added. If cationic polymer is present, it is mixed in a separate container with water at a 1:20 ratio (polymer:water) to form a slurry or dilute solution, which is then added to the cooled composition in the mixing vessel and mixed for 10 minutes. The samples were pH adjusted to 5.5 using citric acid (typically, 0.2-0.5%), HCl and NaOH were used to create samples spanning a pH range from 4.5 to 7.0. Viscosity was adjusted with sodium chloride for some samples. 2 s⁻¹ viscosities were measured using a cone-and-plate geometry for higher viscosity samples, and with a concentric cylinder (Couette) geometry for lower viscosity samples, according to the Rheology method. DI water is added to bring the final volume to 100%. The mixture is mixed until homogeneous (˜10 minutes). The ingredient amounts shown in Table 1 are the amount of active material.

TABLE 1

| Ingredient | 1A | 1B | 1C | 1D | 1E | 1F |
|---|---|---|---|---|---|---|
| SLT ¹ | 0 | 5.85 | 3.6 | 0 | 0 | 6.5 |
| SMLT ² | 0 | 0.65 | 0.4 | 0 | 0 | 0 |
| Sodium C14-16 AOS ³ | 6.5 | 0 | 4.0 | 10.0 | 9.0 | 0 |
| LAPB ⁴ | 6.5 | 6.5 | 5.0 | 0 | 0 | 6.5 |
| CAPB ⁵ | 0 | 0 | 0 | 5.0 | 4.0 | 0 |
| Cocamide MEA ⁶ | 0 | 0 | 0 | 0 | 2.0 | 0 |
| Guar | 0.25 | 0.25 | 0.25 | 0 | 0 | 0.25 |
| hydroxypropyltrimonium | | | | | | |
| chloride ⁷ | | | | | | |
| Polyquaternium-10 ⁸ | 0 | 0 | 0 | 0.40 | 0.40 | 0 |
| Dimethiconol emulsion ⁹ | 0 | 0 | 0 | 0 | 0.25 | 0 |
| Piroctone olamine | 0 | 0 | 0 | 0.50 | 0 | 0.5 |
| Sodium benzoate | 0.75 | 0.75 | 0.75 | 0.45 | 0.75 | 0.75 |
| Sodium salicylate | 0.45 | 0.45 | 0.45 | 0.45 | 0.45 | 0.45 |
| Sodium Chloride | 1.00 | 1.00 | 1.00 | 0 | 0 | 0 |
| Fragrance | 1.10 | 1.10 | 1.10 | 0.50 | 1.10 | 1.0 |
| pH adjuster - | Adjustable | Adjustable | Adjustable | Adjustable | Adjustable | Adjustable |
| citric acid, HCl | to achieve | to achieve | to achieve | to achieve | to achieve | to achieve |
| and/or NaOH | target pH | target pH | target pH | target pH | target pH | target pH |
| Water | QS | QS | QS | QS | QS | QS |
| Ratio of anionic surfactant | 1:1 | 1:1 | 1.6:1 | 2:1 | 1.5:1 | 1:1 |
| to amphoteric/zwitterionic | | | | | | |
| surfactant | | | | | | |
| pH | 5.19 | 5.24 | 4.89 | 5.41 | 5.50 | 4.82 |
| Viscosity (cP) at | 8,557 | 5,988 | 8,980 | 8,822 | 8,890 | 5,072 |
| 2s\hat{ }−1 | | | | | | |

¹ Sodium lauroyl taurate from P&G Chemicals or Innospec.
² Sodium methyl lauroyl taurate from P&G Chemicals
³ Sodium C14-16 alpha olefin sulfonate (Bioterge ®) from Stepan.
⁴ Lauramidopropyl betaine from Syensqo (0.5% NaCl or less).
⁵ Cocamidopropyl betaine (Mackam ™ C37HP from Syensqo), (4.5%-6% NaCl).
⁶ Cocamide MEA (Comperlan ®) from BASF.
⁷ Naternal ® Excel from Syensqo.
⁸ UCARE ® JR30M from Dow.
⁹ Xiameter ™ MEM-1872 from Dow.

### Example 2: Viscosities of Various Compositions as a Function of pH

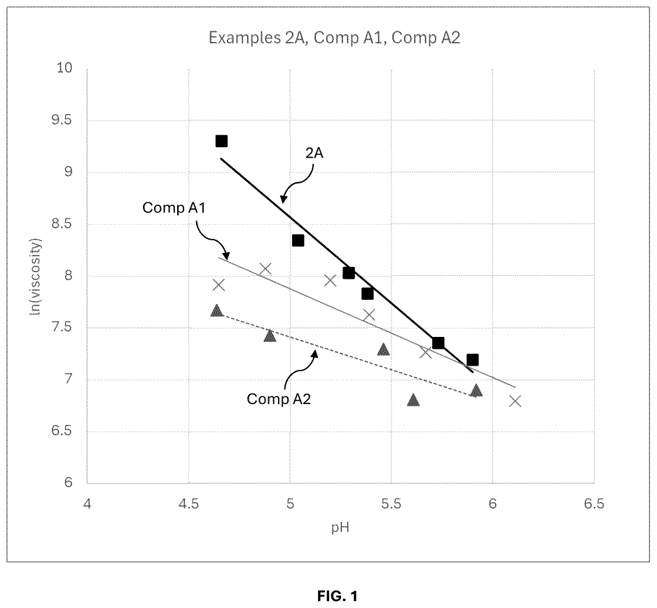

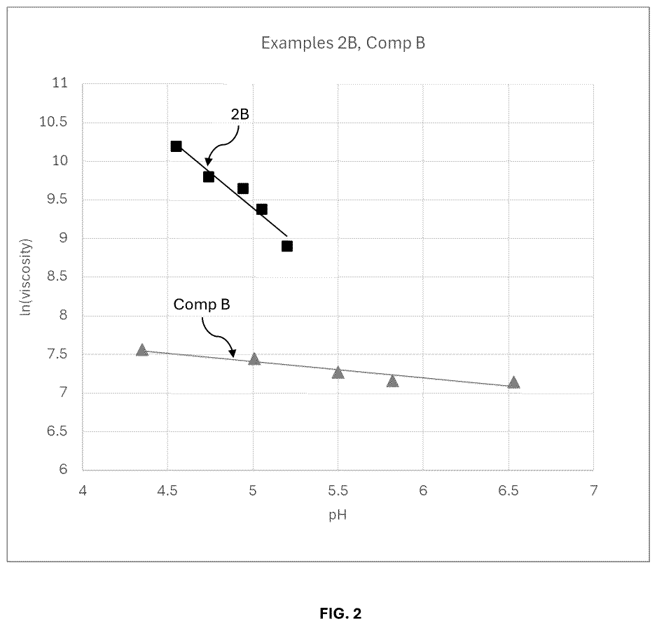

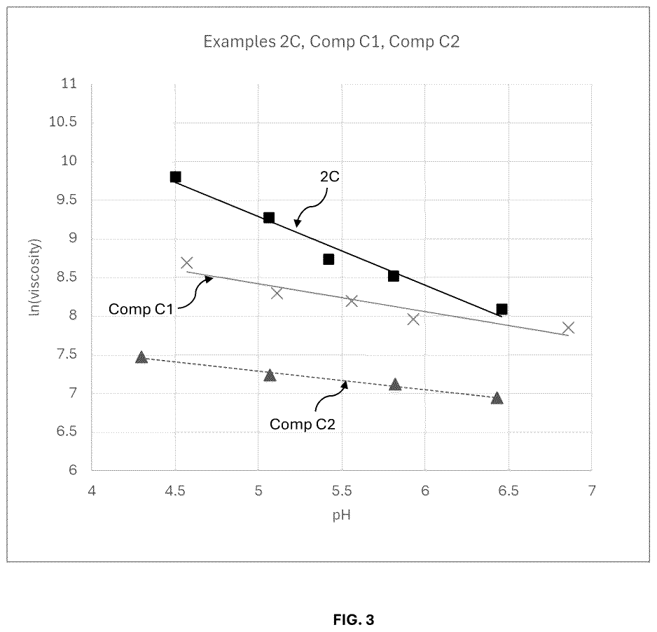

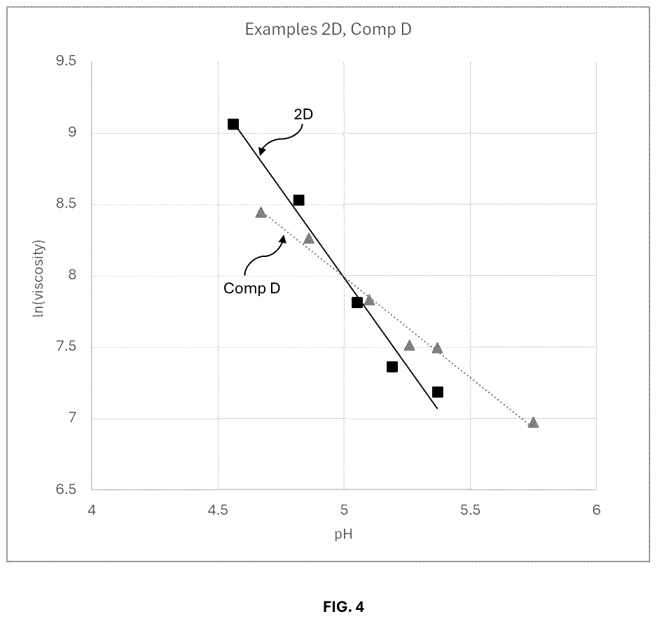

The personal care compositions tested in this example are shown in Table 2. The compositions were adjusted to various pH levels using citric acid, HCl and/or NaOH to create samples spanning a pH range from 4.5 to 7.0. Viscosity was measured according to the Rheology method described above. The results of the testing are illustrated in FIGS. 1, 2, 3, and 4.

TABLE 2

| Ingredient | 2A | Comp A1 | Comp A2 |
|---|---|---|---|
| SLT ¹ | 3.6 | 3.6 | 3.6 |
| SMLT ² | 0.4 | 0.4 | 0.4 |
| Sodium C14-16 AOS ³ | 4 | 4 | 4 |
| LAPB ⁴ | 5 | 0 | 0 |
| Lauryl Glucoside ⁵ | 0 | 5 | 5 |
| PPG-2 Hydroxyethyl cocamide ⁶ | 0 | 0 | 0 |
| Polyquaternium-10 ⁷ | 0 | 0 | 0 |
| Guar hydroxypropyltrimonium chloride ⁸ | 0.25 | 0.25 | 0.25 |
| Dimethiconol Emulsion ⁹ | 0 | 0 | 0.3 |
| Sodium benzoate | 0.75 | 0.75 | 0.75 |
| Sodium salicylate | 0.45 | 0.45 | 0.45 |
| Tetrasodium EDTA | 0 | 0 | 0 |
| NaCl | 1.0 | 1.0 | 1.0 |
| Fragrance | 1.1 | 1.1 | 1.1 |
| pH adjuster - citric acid, HCl and/or NaOH | Adjustable | Adjustable | Adjustable |
| | to achieve | to achieve | to achieve |
| | desired pH | desired pH | desired pH |
| Water | QS | QS | QS |
| Ratio of anionic surfactant to | 1.6:1 | N/A | N/A |
| amphoteric/zwitterionic surfactant | | | |
| Slope (d ln (viscosity)/ d pH | −1.66 | −0.86 | −0.63 |
| pH | 4.5-6.1 | 4.7-6.1 | 4.6-5.9 |

| Ingredient | 2B | Comp B |
|---|---|---|
| SLT ¹ | 0 | 0 |
| SMLT ² | 0 | 0 |
| Sodium C14-16 AOS ³ | 13 | 13 |
| LAPB ⁴ | 9.6 | 0 |
| Lauryl Glucoside ⁵ | 0 | 0 |
| PPG-2 Hydroxyethyl cocamide ⁶ | 0 | 9.6 |
| Polyquaternium-10 ⁷ | 0.1 | 0.1 |
| Guar hydroxypropyltrimonium chloride ⁸ | 0.5 | 0.5 |
| Dimethiconol Emulsion ⁹ | 0.3 | 0.3 |
| Sodium benzoate | 0.5 | 0.5 |
| Sodium salicylate | 0.25 | 0.25 |
| Tetrasodium EDTA | 0.1 | 0.1 |
| NaCl | 0 | 0 |
| Fragrance | 1.1 | 1.1 |
| pH adjuster - citric acid, HCl and/or NaOH | Adjustable | Adjustable |
| | to achieve | to achieve |
| | desired pH | desired pH |
| Water | QS | QS |
| Ratio of anionic surfactant to | 1.4:1 | N/A |
| amphoteric/zwitterionic surfactant | | |
| Slope (d ln (viscosity)/ d pH | −1.83 | −0.21 |
| pH | 4.6-5.2 | 4.4-6.5 |

| Ingredient | 2C | Comp C1 | Comp C2 |
|---|---|---|---|
| SLT ¹ | 0 | 0 | 0 |
| SMLT ² | 0 | 0 | 0 |
| Sodium C14-16 AOS ³ | 13 | 13 | 13 |
| LAPB ⁴ | 9.6 | 0 | 0 |
| Lauryl Glucoside ⁵ | 0 | 9.6 | 0 |
| PPG-2 Hydroxyethyl cocamide ⁶ | 0 | 0 | 9.6 |
| Polyquaternium-10 ⁷ | 0.5 | 0.5 | 0.5 |
| Guar hydroxypropyltrimonium chloride ⁸ | 0 | 0 | 0 |
| Dimethiconol Emulsion ⁹ | 0.3 | 0.3 | 0.3 |
| Piroctone Olamine ¹⁰ | 0 | 0 | 0 |
| Sodium benzoate | 0.5 | 0.5 | 0.5 |
| Sodium salicylate | 0.25 | 0.25 | 0.25 |
| Tetrasodium EDTA | 0.1 | 0.1 | 0.1 |
| NaCl | 0 | 0 | 0 |
| Fragrance | 1.1 | 1.1 | 1.1 |
| pH adjuster - citric acid, HCl and/or NaOH | Adjustable | Adjustable | Adjustable |
| | to achieve | to achieve | to achieve |
| | desired pH | desired pH | desired pH |
| Water | QS | QS | QS |
| Ratio of anionic surfactant to | 1.4:1 | N/A | N/A |
| amphoteric/zwitterionic surfactant | | | |
| Slope (d ln (viscosity)/ d pH | −0.88 | −0.36 | −0.24 |
| pH | 4.5-6.5 | 4.6-6.9 | 4.3-6.4 |

| Ingredient | 2D | Comp D |
|---|---|---|
| SLT ¹ | 6.5 | 6.5 |
| SMLT ² | 0 | 0 |
| Sodium C14-16 AOS ³ | 0 | 0 |
| LAPB ⁴ | 6.5 | 0 |
| Lauryl Glucoside ⁵ | 0 | 6.5 |
| PPG-2 Hydroxyethyl cocamide ⁶ | 0 | 0 |
| Polyquaternium-10 ⁷ | 0 | 0 |
| Guar hydroxypropyltrimonium chloride ⁸ | 0.25 | 0.25 |
| Dimethiconol Emulsion ⁹ | 0 | 0 |
| Piroctone Olamine ¹⁰ | 0.5 | 0.5 |
| Sodium benzoate | 0.75 | 0.75 |
| Sodium salicylate | 0.45 | 0.45 |
| Tetrasodium EDTA | 0.1 | 0.1 |
| NaCl | 0 | 0 |
| Fragrance | 1.0 | 1.0 |
| pH adjuster - citric acid, HCl and/or NaOH | Adjustable | Adjustable |
| | to achieve | to achieve |
| | desired pH | desired pH |
| Water | QS | QS |
| Ratio of anionic surfactant to | 1:1 | N/A |
| amphoteric/zwitterionic surfactant | | |
| Slope (d ln (viscosity)/ d pH | −2.48 | −1.41 |
| pH | 4.6-5.4 | 4.7-5.8 |

¹ Sodium lauroyl taurate from P&G Chemicals or Innospec.
² Sodium methyl lauroyl taurate from P&G Chemicals.
³ Sodium C14-16 alpha olefin sulfonate (Bioterge ®) from Stepan.
⁴ Lauramidopropyl betaine from Syensqo
⁵ Plantaren ® 1200 N UP from BASF
⁶ Promidium ™ CO from Croda Inc.
⁷ UCARE ™ Polymer JR-30M from Dow
⁸ Naternal ® Excel from Syensqo
⁹ Xiameter ™ MEM-1872 from Dow
¹⁰ Octopirox from Clariant

| Ingredient | 2E | 2F | 2G | 2H | 2I | 2J | 2K | 2L |
|---|---|---|---|---|---|---|---|---|
| SLT ¹ | 6.975 | 3.937 | 4.5 | 2.925 | 5.85 | 0 | 0 | 0 |
| SMLT ² | 0.775 | 0.437 | 0.5 | 0.325 | 0.65 | 0 | 0 | 0 |
| Sodium C14-16 AOS ³ | 1 | 4.375 | 3 | 3.25 | 0 | 8.75 | 6.5 | 9 |
| LAPB ⁴ | 4.25 | 4.25 | 5 | 6.5 | 6.5 | 4.25 | 6.5 | 8.5 |
| Guar | 0.25 | 0.25 | 0.25 | 0.25 | 0.25 | 0.25 | 0.25 | 0.15 |
| hydroxypropyltrimonium | | | | | | | | |
| chloride ⁵ | | | | | | | | |
| Piroctone Olamine ⁶ | 0 | 0 | 0 | 0 | 0 | 0 | 0 | 0.5 |
| Sodium benzoate | 0.75 | 0.75 | 0.75 | 0.75 | 0.75 | 0.75 | 0.75 | 0.45 |
| Sodium salicylate | 0.45 | 0.45 | 0.45 | 0.45 | 0.45 | 0.45 | 0.45 | 0 |
| NaCl | 1 | 1 | 1 | 1 | 1 | 1 | 1 | 0 |
| Fragrance | 1.1 | 1.1 | 1.1 | 1.1 | 1.1 | 1.1 | 1.1 | 1.1 |
| Water | QS | QS | QS | QS | QS | QS | QS | QS |
| Ratio of anionic surfactant | 2.1:1 | 2.1:1 | 1.6:1 | 1:1 | 1:1 | 2.1:1 | 1:1 | 1.1:1 |
| to amphoteric/zwitterionic | | | | | | | | |
| surfactant | | | | | | | | |
| Slope (d ln (viscosity)/ d | −1.55 | −1.96 | −1.97 | −1.04 | −1.69 | −1.19 | −1.59 | −1.27 |
| pH | | | | | | | | |
| pH range | 4.7-6.7 | 4.6-5.6 | 4.7-6.1 | 4.5-7.0 | 5.2-6.0 | 4.8-5.6 | 5.0-6.2 | 4.3-5.0 |

¹ Sodium lauroyl taurate from P&G Chemicals.
² Sodium methyl lauroyl taurate from P&G Chemicals.
³ Sodium C14-16 alpha olefin sulfonate (Bioterge ®) from Stepan.
⁴ Lauramidopropyl betaine from Syensqo
⁵ Naternal ® Excel from Syensqo
⁶ Octopirox from Clariant

As shown in Examples 2A to 2L and Comparative A1 to D above (and in FIGS. 1-6), pH, in combination with an amphoteric surfactant, can be used to control viscosity of a personal care composition. The steeper slope (e.g., larger absolute value of slope) shows an enhanced ability to use pH to control viscosity without needing the addition of a thickening polymer.

It was surprisingly found that replacing the amphoteric surfactant of a personal care composition with a non-ionic surfactant significantly impacts the ability to leverage pH to control viscosity as the slope is shallower and thus requires much larger changes in pH to affect an increase in viscosity. For example, Example 2A with 5 wt % amphoteric surfactant (LAPB) had a slope of −1.66 whereas Comparative Example A1 with 5 wt % non-ionic surfactant (Lauryl Glucoside) only had a slope of −0.86. Further, Example 2B with 9.6 wt % LAPB had a slope of −1.83 whereas Comparative Example B with 9.6 wt % PPG-2 Hydroxyethyl cocamide had a slope of only −0.21.

### SLT/SMLT/AOS/LAPB Mixtures 2E 2H, SLT/SMLT/LAPB Mixture 2I

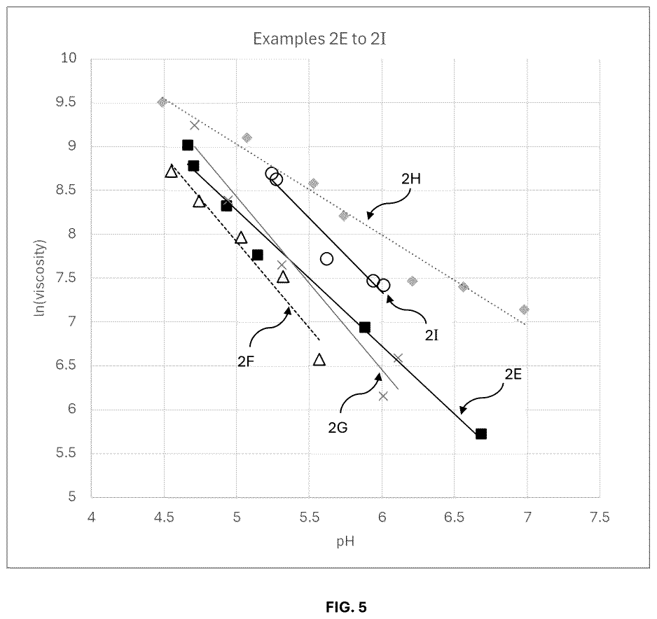

FIG. 5 illustrates the viscosity results obtained for the SLT/SMLT/AOS/LAPB mixtures 2E to 2H and SLT/SMLT/LAPB mixture 2I at various pH. Since viscosities vary over several orders of magnitude, the y-axis shows the natural log of viscosity. The lines in FIG. 5 are linear fits to the data, and the slope values are shown in Table 2. As can be seen in the figures, ln(viscosity) varies almost linearly with pH over the range explored. That the effect of changing pH persists linearly up to pHs of 6.5-7.0 (almost two units above the pKa of LAPB) is surprising because that far above the pKa, substantially more than half of the LAPB would be expected to be deprotonated and therefore have a net neutral charge. With LAPB having mostly a net neutral charge, one would expect it to behave like a non-ionic surfactant and therefore the slope of ln(viscosity) as a function of pH should be non-linear, changing to become much shallower at higher pH. Instead, we find that the slope remains linear and still relatively steep. This surprising relationship facilitates building viscosity efficiently without the addition of a thickening polymer.

### AOS/LAPB Mixtures 2J-2L

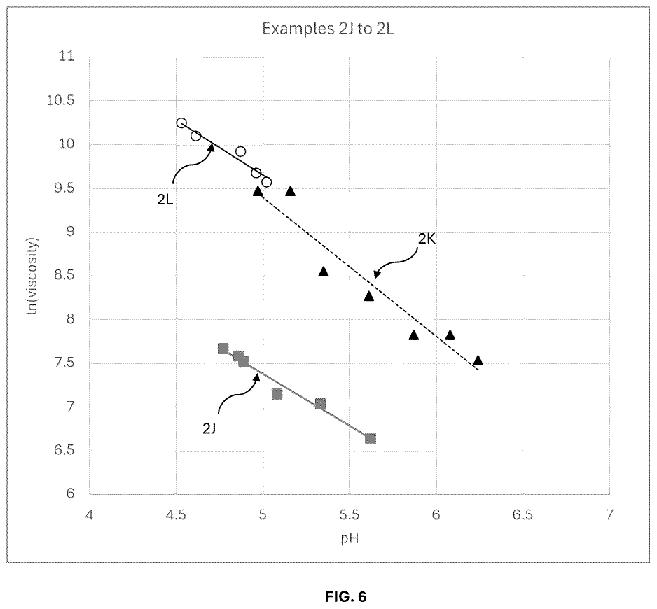

FIG. 6 summarizes the results obtained for the AOS/LAPB mixtures 2J to 2L at various viscosities. The lines are linear fits to the data and the slopes are shown in Table 2. The y-axis shows the natural log of viscosity. Like the SMLT/SLT/AOS/LAPB mixtures, these systems also show a strong pH dependence of viscosity, especially for 1:1 mixtures of AOS/LAPB. The AOS raw material does not contain fatty acids but when combined with LAPB still exhibits viscosity behavior similar to that observed when AOS is combined with SLT, SMLT, and LAPB—reinforcing that protonation of LAPB is primarily responsible for the changing viscosity.

### Example/Combinations

A. A personal care composition, comprising:
a) an anionic surfactant comprising an acyl taurate surfactant, an N-alkyl acyl taurate surfactant, and an alpha olefin sulfonate surfactant, or salts thereof,
b) an amphoteric or zwitterionic surfactant;
c) an aqueous carrier; and
d) 0.3% to 3% of an inorganic salt thickener, wherein the composition has a pH of 3.0 to 6.5, preferably 4 to 6, and a viscosity of 2,500 mPa-s to 25,000 mPa-s, according to the Rheology Method and the slope of ln(viscosity) as a function of pH is less than −0.9, preferably less than −1, more preferably less than −1.5.
   B. The personal care composition of Paragraph A, wherein the acyl taurate surfactant comprises lauroyl taurate or a salt thereof.
   C. The personal care composition of any preceding Paragraph, wherein the alpha olefin sulfonate is a C14-16 alpha olefin sulfonate.
   D. The personal care composition of any preceding Paragraph, wherein the amphoteric or zwitterionic surfactant comprises a betaine.
   E. The personal care composition of any preceding Paragraph, wherein N-alkyl acyl taurate surfactant comprises methyl lauroyl taurate or a salt thereof.
   F. The personal care composition of any preceding Paragraph, wherein the acyl taurate surfactant is present at 60% to 90% by weight, based on the weight of the anionic surfactant.
   G. The personal care composition of any preceding Paragraph, wherein the N-alkyl acyl taurate surfactant is present at 1% to 10% by weight, based on the weight of the anionic surfactant.
   H. The personal care composition of any preceding Paragraph, wherein the amphoteric or zwitterionic surfactant is an amidobetaine, preferably cocamidopropyl betaine, lauramidopropylbetaine or a combination thereof, and more preferably cocamidopropyl betaine.
   I. The personal care composition of any preceding Paragraph, further comprising less than or equal to 3% of a nonionic surfactant, preferably cocamide monoethanolamine.
   J. The personal care composition of any preceding Paragraph, wherein a weight ratio of anionic surfactant to a total amount of amphoteric, zwitterionic and nonionic surfactants is 1:1 to 2:1.
   K. The personal care composition of any preceding Paragraph, wherein the composition is substantially free of an inorganic salt thickener.
   L. The personal care composition of any preceding Paragraph, wherein the composition has a pH of 4 to 6.
   M. The personal care composition of any preceding Paragraph, wherein the amphoteric or zwitterionic surfactant is present at 1% to 20%, preferably 3% to 15%, more preferably 4% to 12% by weight of the composition.
   N. The personal care composition of any preceding Paragraph, wherein the anionic surfactant is present at 5% to 20% by weight of the composition.
   O. The personal care composition of any preceding Paragraph, wherein a total amount of surfactant is 10% to 30%, preferably 12% to 23%, more preferably 13% to 22%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
a) an anionic surfactant comprising an acyl taurate surfactant, an N-alkyl acyl taurate surfactant, and an alpha olefin sulfonate surfactant, or salts thereof,
b) an amphoteric or zwitterionic surfactant;
c) an aqueous carrier; and
d) about 0.3% to about 3% of an inorganic salt thickener,
wherein the composition has a pH of about 3.0 to about 6.5 and a viscosity of about 2,500 mPa-s to 25,000 mPa-s, according to the Rheology Method and the slope of ln(viscosity) as a function of pH is less than −0.9.

2. The personal care composition of claim 1, wherein the acyl taurate surfactant comprises lauroyl taurate or a salt thereof.

3. The personal care composition of claim 1, wherein the alpha olefin sulfonate is a C14-16 alpha olefin sulfonate.

4. The personal care composition of claim 1, wherein the amphoteric or zwitterionic surfactant comprises a betaine.

5. The personal care composition of claim 1, wherein the N-alkyl acyl taurate surfactant comprises methyl lauroyl taurate or a salt thereof.

6. The personal care composition of claim 1, wherein the acyl taurate surfactant is present at about 60% to about 90% by weight, based on the weight of the anionic surfactant.

7. The personal care composition of claim 1, wherein the N-alkyl acyl taurate surfactant is present at about 1% to about 10% by weight, based on the weight of the anionic surfactant.

8. The personal care composition of claim 1, further comprising less than or equal to about 3% by weight of a nonionic surfactant.

9. The personal care composition of claim 8, wherein the nonionic surfactant is cocamide monoethanolamine.

10. The personal care composition of claim 1, wherein a weight ratio of anionic surfactant to a total amount of amphoteric and zwitterionic surfactants is about 1:1 to about 2.1:1.

11. A personal care composition, comprising:
a) an alpha olefin sulfonate surfactant;
b) an amphoteric or zwitterionic surfactant;
c) an aqueous carrier; and
d) about 0.5% to about 5% of an inorganic salt thickener,
wherein the composition has a pH of about 4.0 to about 6.0 and a viscosity of about 2,500 mPa-s to about 25,000 mPa-s, according to the Rheology Method and the slope of ln(viscosity) as a function of pH is less than −0.5.

12. The personal care composition of claim 11, wherein the amphoteric or zwitterionic surfactant comprises a betaine.

13. The personal care composition of claim 11, wherein a weight ratio of anionic surfactant to a total amount of amphoteric and zwitterionic is about 1:1 to about 2.1:1.

14. The personal care composition of claim 11, wherein the alpha olefin sulfonate is a C14-16 alpha olefin sulfonate.

15. A personal care composition, comprising:
a) about 5% to about 20% by weight, based on the weight of the personal care composition of an anionic surfactant comprising an acyl taurate surfactant;
b) about 1% to about 20% by weight, based on the weight of the personal care composition of an amphoteric surfactant;
c) an aqueous carrier; and wherein the composition has a slope of ln(viscosity) as a function of pH less than or equal to about −1.5.

16. The personal care composition of claim 15, wherein the composition is substantially free of an inorganic salt thickener.

17. The personal care composition of claim 15, the acyl taurate surfactant comprises lauroyl taurate or a salt thereof.

18. The personal care composition of claim 15, wherein the composition has a pH of about 4.0 to about 6.0 and a viscosity of about 2,500 mPa-s to about 25,000 mPa-s, according to the Rheology Method.

19. The personal care composition of claim 15, wherein the amphoteric surfactant comprises a betaine.

20. The personal care composition of claim 15, wherein a weight ratio of anionic surfactant to a weight ratio of amphoteric surfactant is about 1:1 to about 2.1:1.
